(19) United States
(12) Patent Application Publication
(10) Pub. No.: US 2012/0219635 A1
(43) Pub. Date: Aug. 30, 2012

(54) Devices and Methods for Tissue Engineering

(75) Inventor: James Jenq Liu, Mason, OH (US)

(73) Assignee: BIO2 TECHNOLOGIES, INC., Woburn, MA (US)

(21) Appl. No.: 13/099,447

(22) Filed: May 3, 2011

Related U.S. Application Data
(60) Provisional application No. 61/331,961, filed on May 6, 2010.

(51) Int. Cl. A61K 33/00; A61K 33/08; B29C 67/20; A61K 33/42

(52) U.S. Cl. 424/601; 424/724; 424/692; 424/722; 424/688; 264/49

(57) ABSTRACT

A bioactive tissue scaffold is fabricated from glass fiber that forms a rigid three-dimensional porous matrix having a bioactive composition. Porosity in the form of interconnected pore space is provided by the pore space between the glass fiber in the porous matrix. Mechanical properties such as strength, elastic modulus, and pore size distribution is provided by the three-dimensional matrix that is formed by bonded overlapping and intertangled fibers. The bioactive tissue scaffold can be formed from raw materials that are not bioactive, but rather precursors to bioactive materials. The bioactive tissue scaffold supports tissue in-growth to provide osteoconductivity as a resorbable tissue scaffold, used for the repair of damaged and/or diseased bone tissue.

### FIELD OF THE INVENTION

The present invention relates generally to the field of porous fibrous medical implants. More specifically, the invention relates to a bioactive fibrous implant having osteostimulative properties in applications of in vivo environments.

### BACKGROUND OF THE INVENTION

Prosthetic devices are often required for repairing defects in bone tissue in surgical and orthopedic procedures. Prostheses are increasingly required for the replacement or repair of diseased or deteriorated bone tissue in an aging population and to enhance the body's own mechanism to produce rapid healing of musculoskeletal injuries resulting from severe trauma or degenerative disease.

Autografting and allografting procedures have been developed for the repair of bone defects. In autografting procedures, bone grafts are harvested from a donor site in the patient, for example from the iliac crest, to implant at the repair site, in order to promote regeneration of bone tissue. However, autografting procedures are particularly invasive, causing risk of infection and unnecessary pain and discomfort at the harvest site. In allografting procedures, bone grafts are used from a donor of the same species but the use of these materials can raise the risk of infection, disease transmission, and immune reactions, as well as religious objections. Accordingly, synthetic materials and methods for implanting synthetic materials have been sought as an alternative to autografting and allografting.

Synthetic prosthetic devices for the repair of defects in bone tissue have been developed in an attempt to provide a material with the mechanical properties of natural bone materials, while promoting bone tissue growth to provide a durable and permanent repair. Knowledge of the structure and bio-mechanical properties of bone, and an understanding of the bone healing process provides guidance on desired properties and characteristics of an ideal synthetic prosthetic device for bone repair. These characteristics include, but are not limited to: bioresorbability so that the device effectively dissolves in the body without harmful side effects; osteostimulation and/or osteoconductivity to promote bone tissue in-growth into the device as the wound heals; and load bearing or weight sharing to support the repair site yet exercise the tissue as the wound heals to promote a durable repair.

Materials developed to date have been successful in attaining at least some of the desired characteristics, but nearly all materials compromise at least some aspect of the bio-mechanical requirements of an ideal hard tissue scaffold.

### BRIEF SUMMARY OF THE INVENTION

The present invention meets the objectives of an effective synthetic bone prosthetic for the repair of bone defects by providing a material that is bioresorbable, osteostimulative, and load bearing. The present invention provides a bioresorbable (i.e., resorbable) tissue scaffold of bioactive glass fiber with a bioactive glass bonding at least a portion of the fiber to form a rigid three dimensional porous matrix. The porous matrix has interconnected pore space with a pore size distribution in the range of about 10 μm to about 500 μm with porosity between 40% and 85% to provide osteoconductivity once implanted in bone tissue. Embodiments of the present invention include pore space having a bi-modal pore size distribution.

Methods of fabricating a synthetic bone prosthesis according to the present invention are also provided that include mixing a glass fiber with a bonding agent, a pore former, and a liquid to provide a plastically formable batch material. In this method, the composition of the glass fiber and the bonding agent are each precursors to a bioactive composition. The formable batch is mixed and kneaded to evenly distribute the glass fiber with the bonding agent, pore former, and binder, and formed into a desired shape. The formed shape is then dried to remove the liquid, and the pore former is removed. The formed shape is then heated to react the glass fiber with the bonding agent to form a porous fiber scaffold having the bioactive composition.

Alternative methods of fabricating a synthetic bone prosthesis according to the present invention are also provided that include the application of a precursor material to a porous fiber scaffold that is then reaction-formed into a bioactive composition.

The method of the present invention generally involves a reaction-formation of a bioactive composition using raw materials that are precursors to the bioactive composition that include fiber precursors, while generally maintaining the form and relative position of the fiber precursors.

These and other features of the present invention will become apparent from a reading of the following descriptions and may be realized by means of the instrumentalities and combinations particularly pointed out in the appended claims.

### BRIEF DESCRIPTION OF THE SEVERAL VIEWS OF THE DRAWING

The foregoing and other objects, features, and advantages of the invention will be apparent from the following detailed description of the several embodiments of the invention, as illustrated in the accompanying drawings in which like reference characters refer to the same parts throughout the different views. The drawings are not necessarily to scale, with emphasis instead being placed upon illustrating the principles of the invention.

FIG. 1 is a ternary phase diagram of soda-lime glass according to the background art.

FIG. 2 is a scanning electron micrograph at approximately 100× magnification showing an embodiment of a bioactive tissue scaffold according to the present invention.

FIG. 3 is a flowchart of an embodiment of a method of the present invention for forming the bioactive tissue scaffold of FIG. 1.

FIG. 4 is a flowchart of an embodiment of a curing step according to the method of FIG. 3.

FIG. 5 is a schematic representation of an embodiment of an object fabricated according to a method of the present invention.

FIG. 6 is a schematic representation of the object of FIG. 5 upon completion of a volatile component removal step of the method of the present invention.

FIG. 7 is a schematic representation of the object of FIG. 6 upon completion of a reaction formation step of the method of the present invention.

FIG. 8 is a flowchart of an alternate embodiment of the present invention for forming the bioactive tissue scaffold of FIG. 1.

FIG. 9 is a side elevation view of a bioactive tissue scaffold according to the present invention manufactured into a spinal implant.

FIG. 10 is a side perspective view of a spine having the spinal implant of FIG. 9 implanted in the intervertebral space.

FIG. 11 is a schematic drawing showing an isometric view of a bioactive tissue scaffold according to the present invention manufactured into an osteotomy wedge.

FIG. 12 is a schematic drawing showing an exploded view of the osteotomy wedge of FIG. 11 operable to be inserted into an osteotomy opening in a bone.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION OF THE INVENTION

The present invention provides a synthetic prosthetic tissue scaffold for the repair of tissue defects. As used herein, the terms “synthetic prosthetic tissue scaffold” and “bone tissue scaffold” and “tissue scaffold” and “synthetic bone prosthetic” in various forms may be used interchangeably throughout. In an embodiment, the synthetic prosthetic tissue scaffold is bioresorbable once implanted in living tissue. In an embodiment, the synthetic prosthetic tissue scaffold is osteoconductive once implanted in living tissue. In an embodiment, the synthetic prosthetic tissue scaffold is osteostimulative once implanted in living tissue. In an embodiment, the synthetic prosthetic tissue scaffold is load bearing once implanted in living tissue.

Various types of synthetic implants have been developed for tissue engineering applications in an attempt to provide a synthetic prosthetic device that mimics the properties of natural bone tissue and promotes healing and repair of tissue. Metallic and bio-persistent structures have been developed to provide high strength in a porous structure that promotes the growth of new tissue. These materials however, are not bioresorbable and must either be removed in subsequent surgical procedures or left inside the body for the life of the patient. A disadvantage of bio-persistent metallic and biocompatible implants is that the high load bearing capability does not transfer to regenerated tissue surrounding the implant. When hard tissue is formed, stress loading results in a stronger tissue but the metallic implant shields the newly formed bone from receiving this stress. Stress shielding of bone tissue therefore results in weak bone tissue which can actually be resorbed by the body, which is an initiator of prosthesis loosening.

Implants into living tissue evoke a biological response dependent upon a number of factors, such as the composition of the implant. Biologically inactive materials are commonly encapsulated with fibrous tissue to isolate the implant from the host. Metals and most polymers produce this interfacial response, as do nearly inert ceramics, such as alumina or zirconia. Biologically active materials or bioactive materials, elicit a biological response that can produce an interfacial bond securing the implant material to the living tissue, much like the interface that is formed when natural tissue repairs itself. This interfacial bonding can lead to an interface that stabilizes the scaffold or implant in the bony bed and provide stress transfer from the scaffold across the bonded interface into the bone tissue. When loads are applied to the repair, the bone tissue including the regenerated bone tissue is stressed, thus limiting bone tissue resorption due to stress shielding. Bioactive materials can exhibit a range of bioactivity: low levels of bioactivity exhibit a slow rate of bonding to living tissue; and high levels of bioactivity exhibit relatively fast rates of bonding to living tissue. A bioresorbable material can elicit the same response as a bioactive material, but can also exhibit complete chemical degradation by body fluid.

The challenge in developing a resorbable tissue scaffold using biologically active and resorbable materials is to attain load bearing strength with porosity sufficient to promote the growth of bone tissue. Conventional bioactive bioglass and bioceramic materials in a porous form are not known to be inherently strong enough to provide load-bearing strength as a synthetic prosthesis or implant. Conventional bioactive materials prepared into a tissue scaffold with sufficient porosity to be osteostimulative have not exhibited load bearing strength. Similarly, conventional bioactive materials in a form that provides sufficient strength do not exhibit a pore structure that can be considered to be osteostimulative.

Fiber-based structures are generally known to provide inherently higher strength to weight ratios, given that the strength of an individual fiber can be significantly greater than powder-based or particle-based materials of the same composition. A fiber can be produced with relatively few discontinuities that contribute to the formation of stress concentrations for failure propagation. By contrast, a powder-based or particle-based material requires the formation of bonds between each of the adjoining particles, with each bond interface potentially creating a stress concentration. Furthermore, a fiber-based structure provides for stress relief and thus, greater strength, when the fiber-based structure is subjected to strain in that the failure of any one individual fiber does not propagate through adjacent fibers. Accordingly, a fiber-based structure exhibits superior mechanical strength properties over an equivalent size and porosity than a powder-based material of the same composition.

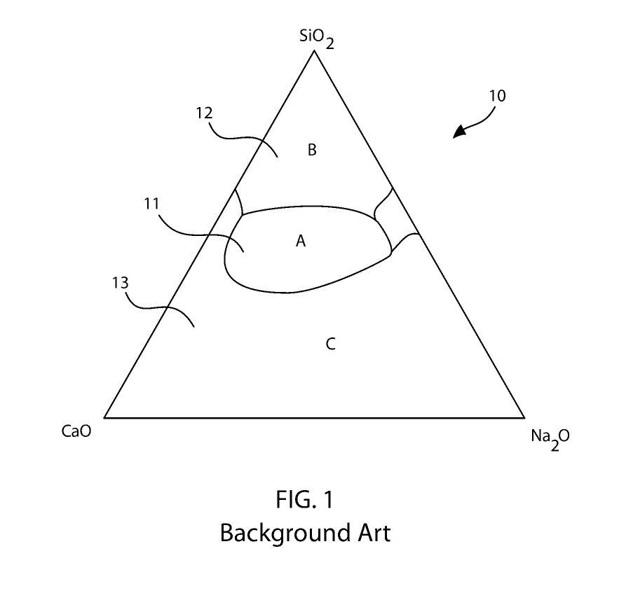

Examples of bioactive glass materials include materials composed of SiO₂, Na₂O, CaO, and P₂O₅ in various ranges of compositions. Other compositions, including B₂O₃ and small amounts of Al₂O₃ and others can be included, with the compositional makeup determining the level of bioactivity and the rate of absorption in vivo. FIG. 1 is a ternary phase diagram for soda lime glass 10 indicating regions for which compositions of SiO₂—CaO—Na₂O have been shown to exhibit bioactivity according to the background art. In FIG. 1, the bioactive region A 11 is a compositional range in which materials have exhibited various degrees of bone bonding and/or resorption indicating bioactivity. The bio-compatible region B 12 is a compositional range in which materials are compatible as an implant in living tissue, but bioactivity has not been observed. Materials within the compositional range of the biocompatible region B 12 are readily formed into a fiber form due to the high silica content. By contrast, the bio-compatible region C 13 is a compositional range that can be compatible as an implant in living tissue, though without exhibiting bioactivity, but these materials are not readily provided in a fiber form. Materials in the bioactive region A 12 can be formed into a fiber if the compositional range is on the high side for the silica component, and the materials cannot be readily formed into a fiber for compositional ranges with lower quantities of silica.

In multi-component systems, such as SiO₂—NaO₂—CaO—P₂O₅—B₂O₃—Al₂O₃ the compositional makeup to bioactivity relationship cannot be expressed in a two-dimensional diagram, such as FIG. 1. Furthermore, the addition of various components, to enhance bioactivity can prevent the ability to readily provide the material in a fiber form. Conversely, the addition of components to enhance the ability to form the material into a fiber, such as, for example, alumina, can reduce the level of bioactivity. Accordingly, the components and constituents of the materials that result in bioactivity can create difficulties in conventional fiber-forming processes and methods.

The present invention provides a fiber-based material for tissue engineering applications that is bioresorbable, with load bearing capability, and osteostimulative with a pore structure that can be controlled and optimized to promote the in-growth of bone, that can be formed from readily obtained fibrous raw materials. A fiber material that is a precursor to a bioactive composition, but not necessarily bioactive in the raw fiber material form, is used to create a fiber-based material that exhibits bioactivity.

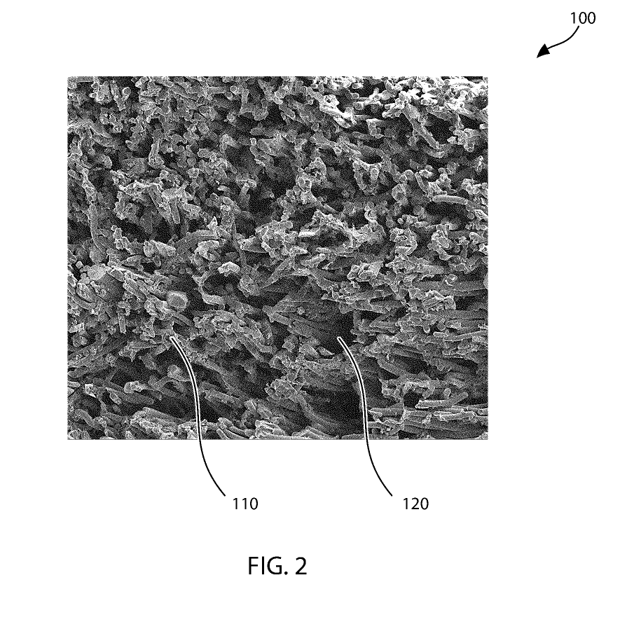

FIG. 2 is an optical micrograph at approximately 100× magnification showing an embodiment of a bioactive tissue scaffold 100 of the present invention. The bioactive tissue scaffold 100 is a rigid three-dimensional matrix 110 forming a structure that mimics bone structure in strength and pore morphology. As used herein, the term “rigid” means the structure does not significantly yield upon the application of stress until it fractured in the same way that natural bone would be considered to be a rigid structure. The scaffold 100 is a porous material having a network of pores 120 that are generally interconnected. In an embodiment, the interconnected network of pores 120 provide osteoconductivity. As used herein, the term osteoconductive means that the material can facilitate the in-growth of bone tissue. Cancellous bone of a typical human has a compressive crush strength ranging between about 4 to about 12 MPa with an elastic modulus ranging between about 0.1 to about 0.5 GPa. As will be shown herein below, the bioactive tissue scaffold 100 of the present invention can provide a porous osteostimulative structure in a bioactive material with porosity greater than 50% and compressive crush strength greater than 4 MPa, up to, and exceeding 22 MPa.

In an embodiment, the three dimensional matrix 110 is formed from fibers that are bonded and fused into a rigid structure, with a composition that exhibits bioresorbability. The use of fibers as a raw material for creating the three dimensional matrix 110 provides a distinct advantage over the use of conventional bioactive or bioresorbable powder-based raw materials. In an embodiment, the fiber-based raw material provides a structure that has more strength at a given porosity than a powder-based structure. In an embodiment, the use of fibers as the primary raw material results in a bioactive material that exhibits more uniform and controlled dissolution rates in body fluid.

In an embodiment, the fiber-based material of the three-dimensional matrix 110 exhibits superior bioresorbability characteristics over the same compositions in a powder-based or particle-based system. For example, dissolution rates are increasingly variable and thus, unpredictable, when the material exhibits grain boundaries, such as a powder-based material form, or when the material is in a crystalline phase. Particle-based materials have been shown to exhibit rapid decrease in strength when dissolved by body fluids, exhibiting failures due to fatigue from crack propagation at the particle grain boundaries. Since bioactive glass or ceramic materials in fiber form are generally amorphous, and the heat treatment processes of the methods of the present invention can better control the amount and degree of ordered structure and crystallinity, the tissue scaffold 100 of the present invention can exhibit more controlled dissolution rates, with higher strength.

The bioactive tissue scaffold 100 of the present invention provides desired mechanical and chemical characteristics, combined with pore morphology to promote osteoconductivity. The network of pores 120 is the natural interconnected porosity resulting from the space between intertangled, nonwoven fiber material in a structure that mimics the structure of natural bone. Furthermore, using methods described herein, the pore size can be controlled, and optimized, to enhance the flow of blood and body fluid within of the scaffold 100 and regenerated bone. For example, pore size and pore size distribution can be controlled through the selection of pore formers and organic binders that are volatilized during the formation of the scaffold 100. Pore size and pore size distribution can be determined by the particle size and particle size distribution of the pore former including a single mode of pore sizes, a bi-modal pore size distribution, and/or a multi-modal pore size distribution. The porosity of the scaffold 100 can be in the range of about 40% to about 85%. In an embodiment, this range promotes the process of osteoinduction of the regenerating tissue once implanted in living tissue while exhibiting load bearing strength.

The scaffold 100 according to the present invention is fabricated using fibers as a raw material that create a bioactive composition. The fibers can be composed of a material that is a precursor to a bioactive material. The term “fiber” as used herein is meant to describe a filament or elongated member in a continuous or discontinuous form having an aspect ratio greater than one, and formed from a fiber-forming process such as drawn, spun, blown, or other similar process typically used in the formation of fibrous materials or high aspect-ratio materials.

Bioactive materials, such as silica- or phosphate-based glass materials with certain compositional modifiers that result in bioactivity, including but not limited to modifiers such as oxides of magnesium, sodium, potassium, calcium, phosphorus, and boron exhibit a narrow working range because the modifiers effectively reduce the devitrification temperature of the bioactive material. The working range of a glass material is typically known to be the range of temperatures at which the material softens such that it can be readily formed. In a glass fiber forming process, the glass material in a billet or frit form is typically heated to a temperature in the working range upon which the glass material is molten and can be drawn or blown into a continuous or discontinuous fiber. The working range of bioactive glass materials is inherently narrow since the devitrification temperature of the glass material is either extremely close or within the working range of the material. In other words, in a typical process for the formation of fiber-based bioactive glass compositions, the temperature at which a fiber can be drawn, blown, or otherwise formed, is close to the devitrification temperature of the bioactive glass composition. When certain bioactive glass materials are drawn or blown into a fiber form at or near the devitrification temperature, the molten or softened glass undergoes a phase change through crystallization that inhibits the continuous formation of fiber.

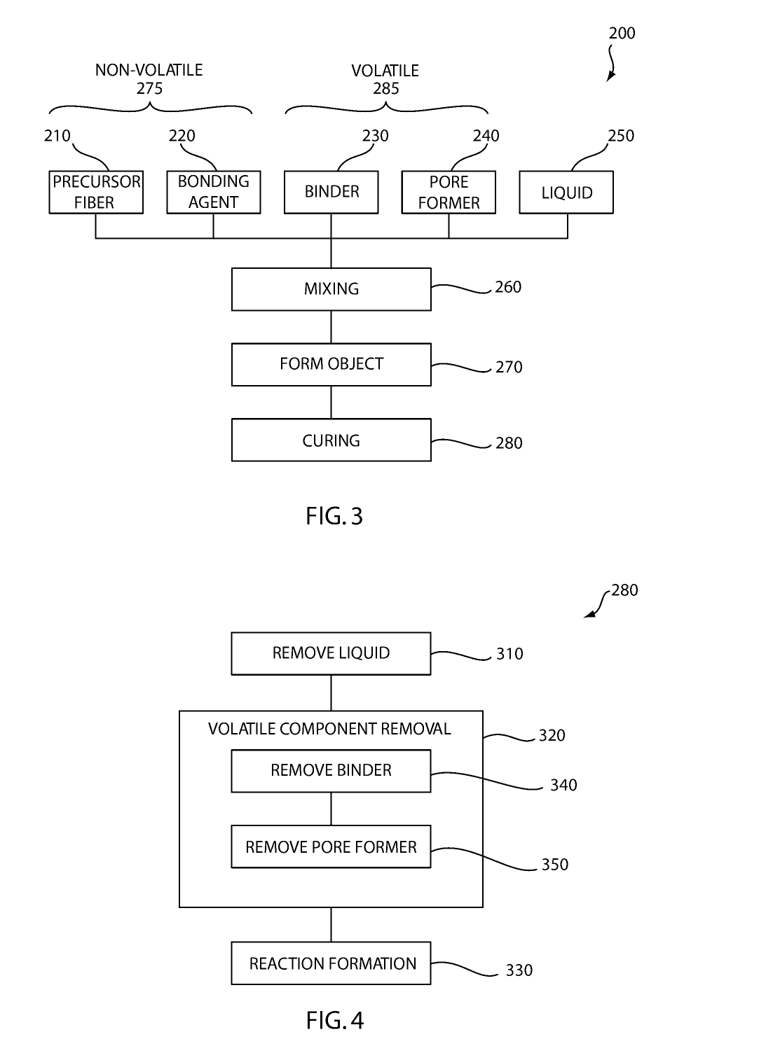

Referring to FIG. 3, an embodiment of a method 200 of forming the bioactive tissue scaffold 100 is shown. As will be described in greater detail below, the method 200 provides for the fabrication of a bioactive tissue scaffold using raw materials including a precursor fiber 210 that are precursors to a bioactive composition that react to form the three-dimensional matrix 110 in a bioactive composition. Generally, bulk precursor fibers 210 are mixed with a bonding agent 220, a binder 230, and a liquid 250 to form a plastically moldable material, which is then cured to form the bioactive tissue scaffold 100. The curing step 280 selectively removes the volatile elements of the mixture, leaving the pore space 120 open and interconnected, and effectively fuses and bonds the fibers 210 into the rigid three-dimensional matrix 110 in a bioactive composition.

The bulk fibers 210 can be provided in bulk form, or as chopped fibers in a composition that is a precursor to a bioactive material. A fiber 210 that is precursor to a bioactive material includes a fiber having a composition that is at least one component of the desired bioactive composition. For example, the fiber 210 can be a silica fiber, or it can be a phosphate fiber, or a combination of any of the compositions used to form the desired bioactive composition. The diameter of the fiber 210 can range from about 1 to about 200 μm and typically between about 5 to about 100 μm. Fibers 210 of this type are can be produced with a relatively narrow and controlled distribution of fiber diameters or depending upon the method used to fabricate the fiber, a relatively broad distribution of fiber diameters can be produced. Bulk fibers 210 of a given diameter may be used, or a mixture of fibers having a range of fiber diameters can be used. The diameter of the fibers 210 will influence the resulting pore size, pore size distribution, strength, and elastic modulus of the porous structure, as well as the size and thickness of the three-dimensional matrix 110, which will influence not only the osteoconductivity of the scaffold 100, but also the rate at which the scaffold 100 is dissolved by body fluids when implanted in living tissue and the resulting strength characteristics, including compressive strength and elastic modulus.

The fibers 210 used according to the present invention as herein described are typically continuous and/or chopped glass fiber. As described herein above certain bioactive glass compositions are difficult to form as a fiber because the working range of the material is extremely narrow. Silica glass in various compositions can be readily drawn into continuous or discontinuous fiber but the addition of calcium oxide and/or phosphate compounds necessary to create a silica-based bioactive composition are the very compounds that result in the reduction of the working range of the silica-based glass. The use of a fiber 210 that has a composition that is a precursor to the desired bioactive composition provides for a readily-obtained and easily formed fiber material to form a porous fiber-based structure that is converted into the desired bioactive composition during the formation of the tissue scaffold.

Examples of fiber 210 that can be used according to the present invention include silica glass or quartz glass fiber. Silica-based materials having a calcium oxide content less than 30% by weight can be typically drawn or blown into fiber form. Silica-based glass materials are generally required to have an alumina content less than 2% by weight since any amount of alumina in excess of that amount will reduce the bioactive characteristics of the resulting structure. Phosphate glasses are precursors to bioactive compositions and can be readily provided in fiber form. These precursor materials that exhibit a sufficient working range can be made into a fiber form through melting in any one of various methods. An exemplary method involves a combination of centrifugal spinning and gaseous attenuation. A glass stream of the appropriate viscosity flows continuously from a furnace onto a spinner plate rotating at thousands of revolutions per minute. Centrifugal forces project the glass outward to the spinner walls containing thousands of holes. Glass passes through the holes, again driven by centrifugal force, and is attenuated by a blast of heated gas before being collected. In another exemplary method, glass in a molten state is heated in a vessel perforated by one or more holes of a given diameter. The molten glass flows and is drawn through these holes; forming individual fibers. The fibers are merged into strands and collected on a mandrel.

Alternative methods for producing materials that are precursors to bioactive compositions in fiber form can be performed at temperatures less than the melting temperature of the precursor materials. For example, a sol-gel fiber drawing method pulls or extrudes a sol-gel solution of the precursor with the appropriate viscosity into a fiber strand that is subsequently heat treated to bind the material into a cohesive fiber. The sol-gel fiber can be formed from a precursor material or a combination of one or more precursor materials that react with each other and/or the bonding agent 220 to create the desired bioactive composition at the reaction formation 330 step, as described in further detail below. Yet other alternative methods can be used to provide a precursor fiber 210. For example, a fiber can be drawn from one precursor composition, such as silica quartz glass, that can be co-drawn into a composite composition of a coated fiber, such as silica quartz glass coated with a magnesia-silicate glass, or a calcium-silicate glass. The co-drawn fiber would provide silica and magnesia or silica and calcium oxide as precursors to a bioactive composition, such as 13-93 glass to form a bioactive composition at the reaction formation 330 step with additional bonding agent 220 including precursors of oxides of magnesium, sodium, potassium, calcium, and phosphorus.

The binder 230 and the liquid 250, when mixed with the fiber 210, create a plastically formable batch mixture that enables the fibers 210 to be evenly distributed throughout the batch, while providing green strength to permit the batch material to be formed into the desired shape in the subsequent forming step 270. An organic binder material can be used as the binder 230, such as methylcellulose, hydroxypropyl methylcellulose (HPMC), ethylcellulose and combinations thereof. The binder 230 can include materials such as polyethylene, polypropylene, polybutene, polystyrene, polyvinyl acetate, polyester, isotactic polypropylene, atactic polypropylene, polysulphone, polyacetal polymers, polymethyl methacrylate, fumaron-indane copolymer, ethylene vinyl acetate copolymer, styrene-butadiene copolymer, acryl rubber, polyvinyl butyral, inomer resin, epoxy resin, nylon, phenol formaldehyde, phenol furfural, paraffin wax, wax emulsions, microcrystalline wax, celluloses, dextrines, chlorinated hydrocarbons, refined alginates, starches, gelatins, lignins, rubbers, acrylics, bitumens, casein, gums, albumins, proteins, glycols, hydroxyethyl cellulose, sodium carboxymethyl cellulose, polyvinyl alcohol, polyvinyl pyrrolidone, polyethylene oxide, polyacrylamides, polyethyterimine, agar, agarose, molasses, dextrines, starch, lignosulfonates, lignin liquor, sodium alginate, gum arabic, xanthan gum, gum tragacanth, gum karaya, locust bean gum, irish moss, scleroglucan, acrylics, and cationic galactomanan, or combinations thereof. Although several binders 230 are listed above, it will be appreciated that other binders may be used. The binder 230 provides the desired rheology and cohesive strength of the plastic batch material in order to form a desired object and maintaining the relative position of the fibers 210 in the mixture while the object is formed, while remaining inert with respect to the bioactive materials. The physical properties of the binder 230 will influence the pore size and pore size distribution of the pore space 120 of the scaffold 100. Preferably, the binder 230 is capable of thermal disintegration, or selective dissolution, without impacting the chemical composition of the bioactive components, including the fiber 210.

The fluid 250 is added as needed to attain a desired rheology in the plastic batch material suitable for forming the plastic batch material into the desired object in the subsequent forming step 270. Water is typically used, though solvents of various types can be utilized. Rheological measurements can be made during the mixing step 260 to evaluate the plasticity and cohesive strength of the mixture prior to the forming step 270.

Pore formers 240 can be included in the mixture to enhance the pore space 120 of the bioactive scaffold 100. Pore formers are non-reactive materials that occupy volume in the plastic batch material during the mixing step 260 and the forming step 270. When used, the particle size and size distribution of the pore former 240 will influence the resulting pore size and pore size distribution of the pore space 120 of the scaffold 100. Particle sizes can typically range between about 25 μm or less to about 450 μm or more, or alternatively, the particle size for the pore former can be a function of the fibers 210 diameter ranging from about 0.1 to about 100 times the diameter of the fibers 210. The pore former 240 must be readily removable during the curing step 280 without significantly disrupting the relative position of the surrounding fibers 210. In an embodiment of the invention, the pore former 240 can be removed via pyrolysis or thermal degradation, or volatilization at elevated temperatures during the curing step 280. For example, microwax emulsions, phenolic resin particles, flour, starch, or carbon particles can be included in the mixture as the pore former 240. Other pore formers 240 can include carbon black, activated carbon, graphite flakes, synthetic graphite, wood flour, modified starch, celluloses, coconut shell husks, latex spheres, bird seeds, saw dust, pyrolyzable polymers, poly(alkyl methacrylate), polymethyl methacrylate, polyethyl methacrylate, poly n-butyl methacrylate, polyethers, poly tetrahydrofuran, poly(1,3-dioxolane), poly(alkalene oxides), polyethylene oxide, polypropylene oxide, methacrylate copolymers, polyisobutylene, polytrimethylene carbonate, polyethylene oxalate, polybeta-propiolactone, polydelta-valerolactone, polyethylene carbonate, polypropylene carbonate, vinyl toluene/alpha-methylstyrene copolymer, styrene/alpha-methyl styrene copolymers, and olefin-sulfur dioxide copolymers. Pore formers 240 may be generally defined as organic or inorganic, with the organic typically burning off at a lower temperature than the inorganic. Although several pore formers 240 are listed above, it will be appreciated that other pore formers 240 may be used. Pore formers 240 can be, though need not be, fully biocompatible since they are removed from the scaffold 100 during processing.

Additional precursors to the desired bioactive material can be provided as a bonding agent 220 to combine with the composition of the fiber 210 to form the bioactive composition of the three-dimensional matrix 110 and to promote strength and performance of the resulting bioactive scaffold 100. The bonding agent 220 can include powder-based material of the same composition as the bulk fiber 210, or it can include powder-based material of a different composition. In an embodiment of the invention the bonding agent 220 can be coated on the fibers 210 as a sizing or coating. In this embodiment, additional precursors to the bioactive composition are added to the fiber, for example, as a sizing or coating. In an alternate embodiment, the bonding agent 220 is a sizing or coating that is added to the fiber during or prior to the mixing step 260. The bonding agent 220 can be solids dissolved in a solvent or liquid that are deposited on the fiber and/or other bonding agent 220 precursors when the liquid or solvent is removed. As will be explained in further detail below, the bonding agent 220 based additives enhance the bonding strength of the intertangled fibers 210 forming the three-dimensional matrix 110 through the formation of bonds between adjacent and intersecting fibers 210 when the bonding agent 220 reacts with the fiber 210 to form the desired bioactive composition. The relative quantities of the fiber 210 and the bonding agent 220 generally determine the resulting composition of the three-dimensional matrix 110.

The relative quantities of the respective materials, including the bulk fiber 210, the binder 230, and the liquid 250 depend on the overall porosity desired in the bioactive tissue scaffold 100. For example, to provide a scaffold 100 having approximately 60% porosity, the nonvolatile components 275, such as the fiber 210, would amount to approximately 40% of the mixture by volume. The relative quantity of volatile components 285, such as the binder 230 and the liquid 250 would amount to approximately 60% of the mixture by volume, with the relative quantity of binder to liquid determined by the desired rheology of the mixture. Furthermore, to produce a scaffold 100 having porosity enhance by the pore former 240, the amount of the volatile components 285 is adjusted to include the volatile pore former 240. Similarly, to produce a scaffold 100 having strength enhanced by the bonding agent 220, the amount of the nonvolatile components 275 would be adjusted to include the nonvolatile bonding agent 220. It can be appreciated that the relative quantities of the nonvolatile components 275 and volatile components 285 and the resulting porosity of the scaffold 100 will vary as the material density may vary due to the reaction of the components during the curing step 280. Specific examples are provided herein below.

In the mixing step 260, the fiber 210, the binder 230, the liquid 250, the pore former 240 and/or the bonding agent 220, if included, are mixed into a homogeneous mass of a plastically deformable and uniform mixture. The mixing step 260 may include dry mixing, wet mixing, shear mixing, and kneading, which can be necessary to evenly distribute the material into a homogeneous mass while imparting the requisite shear forces to break up and distribute or de-agglomerate the fibers 210 with the non-fiber materials. The amount of mixing, shearing, and kneading, and duration of such mixing processes depends on the selection of fiber's 210 and non-fiber materials, along with the selection of the type of mixing equipment used during the mixing step 260, in order to obtain a uniform and consistent distribution of the materials within the mixture, with the desired rheological properties for forming the object in the subsequent forming step 270. Mixing can be performed using industrial mixing equipment, such as batch mixers, shear mixers, and/or kneaders. The kneading element of the mixing step 260 distributes the fiber 210 with the bonding agent 220 and the binder 230 to provide a formable batch of a homogeneous mass with the fiber being arranged in an overlapping and intertangled relationship with the remaining fiber in the batch.

The forming step 270 forms the mixture from the mixing step 260 into the object that will become the bioactive tissue scaffold 100. The forming step 270 can include extrusion, rolling, pressure casting, or shaping into nearly any desired form in order to provide a roughly shaped object that can be cured in the curing step 280 to provide the scaffold 100. It can be appreciated that the final dimensions of the scaffold 100 may be different than the formed object at the forming step 270, due to expected shrinkage of the object during the curing step 280, and further machining and final shaping may be necessary to meet specified dimensional requirements. In an exemplary embodiment to provide samples for mechanical and in vitro and in vivo testing, the forming step 270 extrudes the mixture into a cylindrical rod using a piston extruder forcing the mixture through a round die.

The object is then cured into the bioactive tissue scaffold 100 in the curing step 280, as further described in reference to FIG. 4. In the embodiment shown in FIG, 4, the curing step 280 can be performed as the sequence of three phases: a drying step 310; a volatile component removal step 320; and a reaction formation step 330. In the first phase, drying 310, the formed object is dried by removing the liquid using slightly elevated temperature heat with or without forced convection to gradually remove the liquid. Various methods of heating the object can be used, including, but not limited to, heated air convection heating, vacuum freeze drying, solvent extraction, microwave or electromagnetic/radio frequency (RF) drying methods. The liquid within the formed object is preferably not removed too rapidly to avoid drying cracks due to shrinkage. Typically, for aqueous based systems, the formed object can be dried when exposed to temperatures between about 90° C. and about 150° C. for a period of about one hour, though the actual drying time may vary due to the size and shape of the object, with larger, more massive objects taking longer to dry. In the case of microwave or RF energy drying, the liquid itself, and/or other components of the object, adsorb the radiated energy to more evenly generate heat throughout the material. During the drying step 310, depending on the selection of materials used as the volatile components, the binder 230 can congeal or gel to provide greater green strength to provide rigidity and strength in the object for subsequent handling.

Once the object is dried, or substantially free of the liquid component 250 by the drying step 310, the next phase of the curing step 280 proceeds to the volatile component removal step 320. This phase removes the volatile components (e.g., the binder 230 and the pore former 240) from the object leaving only the non-volatile components that form the three-dimensional matrix 110 of the tissue scaffold 100. The volatile components can be removed, for example, through pyrolysis or by thermal degradation, or solvent extraction. The volatile component removal step 320 can be further parsed into a sequence of component removal steps, such as a binder burnout step 340 followed by a pore former removal step 350, when the volatile components 285 are selected such that the volatile component removal step 320 can sequentially remove the components. For example, HPMC used as a binder 230 will thermally decompose at approximately 300° C. A graphite pore former 220 will oxidize into carbon dioxide when heated to approximately 600° C. in the presence of oxygen. Similarly, flour or starch, when used as a pore former 220, will thermally decompose at temperatures between about 300° C. and about 600° C. Accordingly, the formed object composed of a binder 230 of HPMC and a pore former 220 of graphite particles, can be processed in the volatile component removal step 320 by subjecting the object to a two-step firing schedule to remove the binder 230 and then the pore former 220. In this example, the binder burnout step 340 can be performed at a temperature of at least about 300° C. but less than about 600° C. for a period of time. The pore former removal step 350 can then be performed by increasing the temperature to at least about 600° C. with the inclusion of oxygen into the heating chamber. This thermally-sequenced volatile component removal step 320 provides for a controlled removal of the volatile components 285 while maintaining the relative position of the non-volatile components 275 in the formed object.

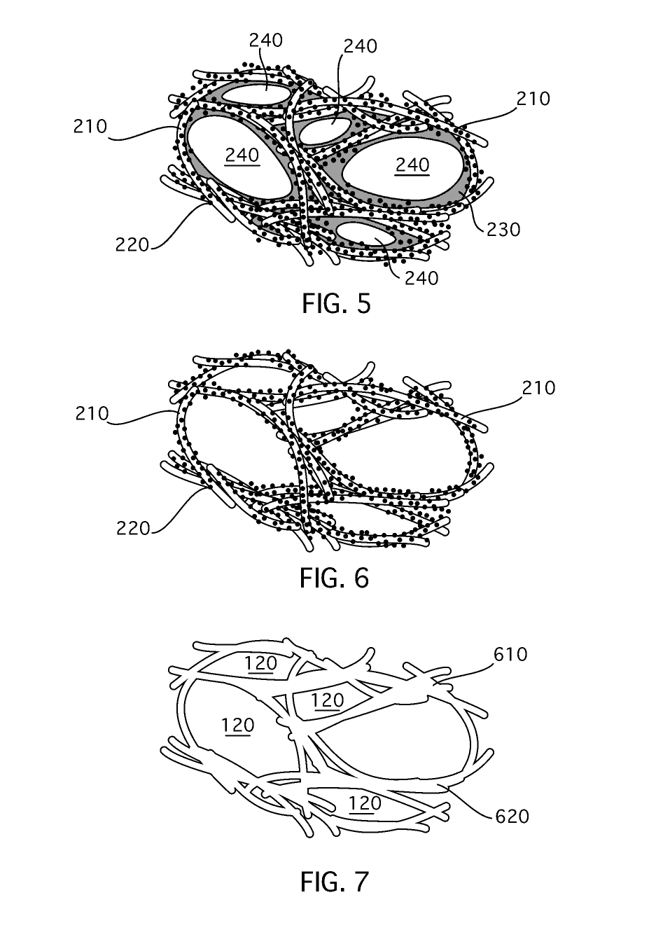

FIG. 5 depicts a schematic representation of the various components of the formed object prior to the volatile component removal step 320. The fibers 210 are intertangled within a mixture of the bonding agent 220, binder 230 and the pore former 240. FIG. 6 depicts a schematic representation of the formed object upon completion of the volatile component removal step 320. The fibers 210 and bonding agent 220 maintain their relative position as determined from the mixture of the fibers 210 with the volatile components 285 as the volatile components 285 are removed. Upon completion of the removal of the volatile components 285, the mechanical strength of the object may be somewhat fragile, and handling of the object in this state should be performed with care. In an embodiment, each phase of the curing step 280 is performed in the same oven or kiln. In an embodiment, a handling tray is provided upon which the object can be processed to minimize handling damage.

FIG. 7 depicts a schematic representation of the formed object upon completion of the last step of the curing step 280, reaction formation 330. Pore space 120 is created between the bonded and intertangled fibers where the binder 230 and the pore former 240 were removed, and the fibers 210 and bonding agent 220 are fused and bonded into the three dimensional matrix 110. The characteristics of the volatile components 285, including the size of the pore former 240 and/or the distribution of particle sizes of the pore former 240 and/or the relative quantity of the binder 230, together cooperate to predetermine the resulting pore size, pore size distribution, and pore interconnectivity of the resulting tissue scaffold 100. The bonding agent 220 and the glass bonds that form at overlapping nodes 610 and adjacent nodes 620 of the three dimensional matrix 110 provide for structural integrity of the resulting three-dimensional matrix 110 having a bioactive composition.

Referring back to FIG. 4, the reaction formation step 330 converts the nonvolatile components 275, including the bulk fiber 210, into the rigid three-dimensional matrix 110 having a bioactive composition as the tissue scaffold 100 while maintaining the pore space 120 created by the removal of the volatile components 275 and maintaining the relative positioning of the fiber 210. The reaction formation step 330 heats the non-volatile components 275 to a temperature upon which the bulk fibers 210 react with the bonding agent 220 to form the bioactive composition and bond to adjacent and overlapping fibers 210, and for a duration sufficient for the reaction to occur and to form the bonds, without melting the fibers 210 or otherwise destroying the relative positioning of the non-volatile components 275. The reaction and bond formation temperature and duration depends on the chemical composition of the non-volatile components 275, including the bulk fiber 210. A bioactive glass fiber or powder of a particular composition exhibits softening and a capability for plastic deformation without fracture at a glass transition temperature. Glass materials typically have a devitrification temperature upon which the amorphous glass structure crystallizes. In an embodiment of the invention, the reaction and bond formation temperature in the reaction formation step 330 is in the working range between the glass transition temperature and the devitrification temperature of the precursors to the bioactive material. For example where precursors to the 13-93 bioactive glass composition are used to form the 13-93 bioactive composition, the reaction temperature can be above the glass transition temperature of about 606° C. and less than the devitrification temperature of about 1,140° C.

In the reaction formation step 330, the formed object is heated to the reaction and bond formation temperature resulting in the formation of glass bonds at overlapping nodes 610 and adjacent nodes 620 of the fiber structure. The bonds are formed at overlapping nodes 610 and adjacent nodes 620 of the fiber structure through a reaction of the bonding agent 220 that flows around the fibers 210, reacting with the fibers 210 to form the bioactive composition including a glass coating and/or glass bonds. In the reaction formation step 330, the material of the fibers 210 participates in a chemical reaction with the bonding agent 220. Further still, the bulk fibers 210 may be a mixture of fiber compositions, with a portion, or all of the fibers 210 participating in a reaction forming bonds to create the three-dimensional matrix 110 in a bioactive composition.

The duration of the reaction formation step 330 depends on the temperature profile during the reaction formation step 330, in that the time at the reaction and bond formation temperature of the fibers 210 is limited to a relatively short duration so that the relative position of the non-volatile components 275, including the bulk fibers 210, does not significantly change. The pore size, pore size distribution, and interconnectivity between the pores in the formed object are determined by the relative position of the bulk fibers 210 by the volatile components 285. While the volatile components 285 are likely burned out of the formed object by the time the bond formation temperature is attained, the relative positioning of the fibers 210 and non-volatile components 275 are not significantly altered. The formed object will likely undergo slight or minor densification during the reaction formation step 330, but the control of pore size and distribution of pore sizes can be maintained, and therefore predetermined by selecting a particle size for the pore former 240 that is slightly oversize or adjusting the relative quantity of the volatile components 285 to account for the expected densification.

In an embodiment of the invention, the bonding agent 220 is a precursor to a bioactive material in a fine powder or nano-particle (e.g., 1-100 nanometers) form. In this embodiment, the small particle sizes react more quickly with the fiber 210 in the reaction formation step 330. In an embodiment of the invention, the reaction between the bonding agent 220 and the fiber 210 also forms a glass that covers and bonds the overlapping nodes 610 and adjacent nodes 620 of the fiber structure before the fiber material is appreciably affected by the exposure to the reaction temperature at or near its glass transition temperature. In this embodiment, for the bonding agent 220 to be more reactive than the bulk fibers 210, the particle size can be in the range of 1 to 1000 times smaller than the diameter of the fibers 210, for example, in the range of 10 microns to 10 nanometers when using 10 micron diameter bulk fibers 210. Nanoparticle sized powder can be produced by milling bioactive glass material in a milling or comminution process, such as impact milling or attrition milling in a ball mill or media mill.

The temperature profile of the reaction formation step 330 can be controlled to control the amount of crystallization and/or minimize the devitrification of the resulting three-dimensional matrix 110. As described above, bioactive glass and bioresorbable glass compounds exhibit more controlled and predictable dissolution rates in living tissue when the amount of accessible grain boundaries of the materials is minimized. These bioactive and bioresorbable materials exhibit superior performance as a bioactive device due to the amorphous structure of the material when fabricated into fibers 210, and the controlled degree of crystallinity that occurs during the heat treatment processing during the bond formation step 330. Therefore, in an embodiment of the method of the present invention, the temperature profile of the reaction formation step 330 is adapted to form the bioactive composition and bond the fiber structure without increasing grain boundaries in the non-volatile materials 275.

In an embodiment of the method of the present invention, the reaction and bond formation temperature exceeds the devitrification temperature of the bulk fibers 210 during the bond formation step 330. Resulting compositions of bioactive glass from the precursors can exhibit a narrow working range between its glass transition temperature and the crystallization temperature. In this embodiment, the crystallization of the resulting structure may not be avoided in order to promote the formation of the bioactive composition and the formation of bonds between overlapping and adjacent nodes of the fibers 210 in the structure. For example, bioactive glass in the 45S5 composition has an initial glass transition temperature of about 550° C. and a devitrification temperature of about 580° C. with crystallization temperatures of various phases at temperatures at about 610, about 800, and about 850° C. With such a narrow working range, the formation of the 45S5 composition may be difficult to perform, and as such, the reaction and bond formation temperature may require temperatures in excess of about 900° C. to form the structure. In an alternative embodiment, the reaction and bond formation temperature can exceed the crystallization temperature of at least a portion of the precursors to the bioactive composition, yet still fall within the working range of the remaining precursor materials. In this embodiment, the fibers 210 of a first precursor composition may crystallize, with glass bonds of a second precursor composition forming at overlapping and adjacent nodes of the fiber structure during the formation of the bioactive composition. For example a 13-93 composition in a powder form as a bonding agent 220 can be used with bioactive glass fibers in a 45S5 composition, to form a glass bond above the glass transition temperature of the 13-93 composition but less than the devitrification temperature of the 13-93 composition but exceeds the devitrification temperature of the 45S5 glass fiber composition to form a composite formed object.

In an embodiment of the invention, the temperature profile of the reaction formation step 330 is configured to reach a reaction and bond formation temperature quickly and briefly, with rapid cooling to avoid devitrification of the resulting bioactive material. Various heating methods can be utilized to provide this heating profile, such as forced convection in a kiln, heating the object directly in a flame, laser, or other focused heating methods. In this embodiment, the focused heating method is a secondary heating method that supplements a primary heating method, such as a kiln or oven heating apparatus. The secondary heating method provides the brief thermal excursion to the bond formation temperature, with a fast recovery to a temperature less than the glass transition temperature in order to avoid devitrification of the resulting three-dimensional matrix 110.

In an embodiment of the invention, combustion of the pore former 240 can be used to provide rapid and uniform heating throughout the object during the bond formation step 330. In this embodiment, the pore former removal step 350 generally occurs during the reaction formation step 330. The pore former 240 is a combustible material, such as carbon or graphite, starch, organics or polymers, such as polymethyl methacrylate, or other material that exothermically oxidizes at elevated temperatures less than or equal to the devitrification temperature of the bioactive glass fiber material 210. Generally, the pore former 240 is selected based on the temperature at which the material initiates combustion, as can be determined by thermal analysis, such as Thermogravimetric Analysis (TGA) or Differential Thermal Analysis (DTA), or a combination of TGA and DTA, such as a simultaneous DTA/TGA which detects both mass loss and thermal response. For example, Table 1 shows the results of a DTA/TGA analysis of various materials to determined the exothermic combustion point of the material.

TABLE 1

| Pore Former | Combustion Temperature |
|---|---|
| Activated Carbon | 621° C. |
| Graphite Flakes | 603° C. |
| HPMC | 375° C. |
| PMMA | 346° C. |
| Wood Flour | 317° C. |
| Corn Starch | 292° C. |

During the curing step 280, adapted so the pore former removal step 350 generally occurs during the reaction formation step 330, the pore former combustion increases the temperature of the formed object substantially uniformly and at an increased rate throughout the object. In this way the desired bond formation temperature can be attained reasonably quickly. Once the pore former is fully combusted, the internal temperature of the formed article will decrease because of the thermal gradient between the internal temperature of the formed object resulting from the pore former combustion and the temperature of the surrounding environment in the kiln or oven. The result is that the thermal profile of the curing process 280 can include a sharp and brief thermal excursion at or near the devitrification temperature of the resulting bioactive composition of the three-dimensional matrix 110.

Additional control over the curing step 280 can be provided by controlling the environment of the kiln. For example, inert or stagnant air in the kiln or oven environment can delay the point at which the volatile components 285 are removed or control the rate at which the volatile components are removed. Furthermore, the pore former removal step 340 can be further controlled by the environment by purging with an inert gas, such as nitrogen, until the temperature is greater than the combustion temperature of the pore former, and nearly that of the desired reaction and bond formation temperature. Oxygen can be introduced at the high temperature, so that when the pore former oxidizes, the temperature of the non-volatile materials can be locally increased at or above the glass transition temperature of the precursors, or at or above the reaction and bond formation temperature, until the pore former is fully combusted. At that point, the temperature can be reduced to avoid devitrification and/or the growth of grain boundaries of and within the resulting structure.

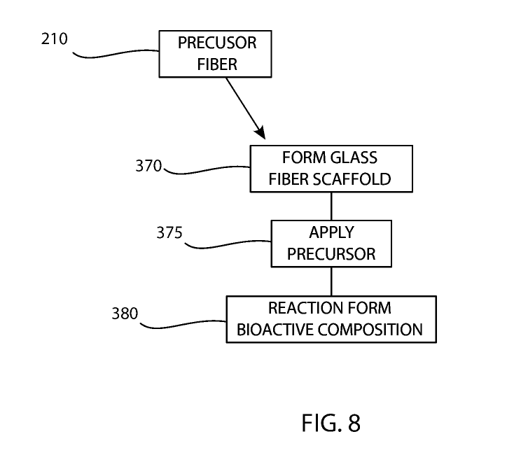

Referring now to FIG. 8, an alternate embodiment of the present invention is shown. In this embodiment, an alternative method 360 provides a fiber-based tissue scaffold formed from precursor fiber 210. As shown in FIG. 8, the precursor fiber 210 is used to form a glass fiber scaffold at step 370, where the precursor is then applied at step 375, which is then reaction formed into a bioactive composition at step 380.

In this alternative method 360, the forming step 370 can be similar to the method described above with reference to FIG. 3 and FIG. 4 wherein the resulting scaffold is not fully converted into a bioactive composition or converted into a bioactive composition that has a low level of bioactivity. In other words, at forming step 370 the precursor fiber 210 and any additives that may be utilized to form the glass fiber scaffold does not fully convert into a bioactive scaffold. The post-processing of application step 375 applies the precursor materials that can fully convert the scaffold material into a bioactive composition, or increase the bioactivity of the scaffold material, at the reaction step 380. Alternatively, the forming step 370 can be sintered bulk precursor fiber 210 to form a scaffold material, though this method would not provide control of pore size distribution and other characteristics that can be provided by the method described above with reference to FIG. 3 and FIG. 4.

The apply precursor step 375 can be performed in any number of methods to introduce a precursor to the glass fiber scaffold produced at step 370. For example, the precursor can be in a colloidal solution that can be immersion applied to the scaffold, or vacuum drawn into the porous matrix of the fiber scaffold. Alternatively, the precursor can be in liquid form or dissolved in a solvent that can be applied by immersion followed by drying. Still more examples include chemical vapor deposition of the precursor or other gas phase deposition of precursor compositions.

The reaction step 380 can be heating the precursor glass fiber with applied precursors in a kiln or furnace to a reaction formation temperature for a duration of time sufficient for the applied precursors to react with the precursor fiber to form the desired bioactive composition. In this reaction step 380, the precursors applied at step 375 react with the precursor fiber 210 to form the bioactive composition.

In an example of the alternative method 360, a calcium-silica glass fiber having approximately 27.4% calcium and 72.6% silica is the precursor fiber 210 that can be readily fabricated in a continuous fiber form. The calcium-silica glass fiber is used to form a three-dimensional porous matrix by sintering the calcium-silica fiber in chopped form to approximately 655° C. for about 30 minutes and cooled to form a glass fiber scaffold. A colloidal solution of precursors of oxides of sodium (22% Na₂O), magnesium (19% MgO), phosphorus (14.8% P₂O₅), and potassium (44.4% K₂O) are applied to load approximately 27% solids of the precursors to the calcium-silica glass fiber scaffold and dried. The scaffold with the precursors applied are fired in a stagnant air kiln at 800° C. for approximately 60 minutes for the precursors to react with the calcium-silica glass fiber to form a bioactive composition having a uniform composition of 53% SiO₂, 5% MgO, 6% Na₂O, 12% K₂O, 20% CaO, and 4% P₂O₅ (by weight).

In an embodiment of the present invention, the strength and durability of the tissue scaffold 100 can be enhanced by annealing the formed object subsequent to or during the curing step 280. During the reaction formation step 330 when the non-volatile components 275 are heated to the reaction and bond formation temperature and subsequently cooled, thermal gradients within the materials may occur during a subsequent cooling phase. Thermal gradients in the material during cooling may induce internal stress that pre-loads the structure with stress that effectively reduces the amount of external stress the object can endure before mechanical failure. Annealing the tissue scaffold 100 involves heating the object to a temperature that is the stress relief point of the material, i.e., a temperature at which the glass material is still hard enough to maintain its shape and form, but enough for any internal stress to be relieved. The annealing temperature is determined by the composition of the resulting structure (i.e., the temperature at which the viscosity of the material softens to stress relief point), and the duration of the annealing process is determined by the relative size and thickness of the internal structure (i.e. the time at which the temperature reaches steady state throughout the object). The annealing process cools slowly at a rate that is limited by the heat capacity, thermal conductivity, and thermal expansion coefficient of the material. In an exemplary embodiment of the present invention, a fourteen millimeter diameter extruded cylinder of a porous bioactive tissue scaffold having a 13-93 composition can be annealed by heating the object in a kiln or oven at 500° C. for six hours and cooled to room temperature over approximately four hours.

The bioactive tissue scaffolds of the present invention can be used in procedures such as an osteotomy (for example in the hip, knee, hand and jaw), a repair of a structural failure of a spine (for example, an intervertebral prosthesis, lamina prosthesis, sacrum prosthesis, vertebral body prosthesis and facet prosthesis), a bone defect filler, fracture revision surgery, tumor resection surgery, hip and knee prostheses, bone augmentation, dental extractions, long bone arthrodesis, ankle and foot arthrodesis, including subtalar implants, and fixation screws pins. The bioactive tissue scaffolds of the present invention can be used in the long bones, including, but not limited to, the ribs, the clavicle, the femur, tibia, and fibula of the leg, the humerus, radius, and ulna of the arm, metacarpals and metatarsals of the hands and feet, and the phalanges of the fingers and toes. The bioactive tissue scaffolds of the present invention can be used in the short bones, including, but not limited to, the carpals and tarsals, the patella, together with the other sesamoid bones. The bioactive tissue scaffolds of the present invention can be used in the other bones, including, but not limited to, the cranium, mandible, sternum, the vertebrae and the sacrum. In an embodiment, the tissue scaffolds of the present invention have high load bearing capabilities compared to conventional devices. In an embodiment, the tissue scaffolds of the present invention require less implanted material compared to conventional devices. Furthermore, the use of the tissue scaffold of the present invention requires less ancillary fixation due to the strength of the material. In this way, the surgical procedures for implanting the device are less invasive, more easily performed, and do not require subsequent surgical procedures to remove instruments and ancillary fixations.

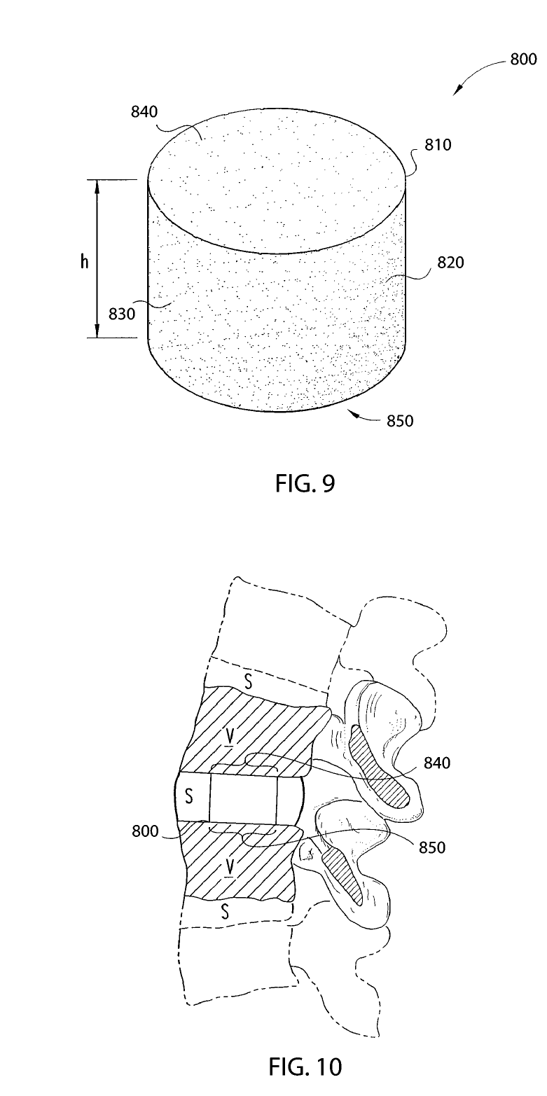

In one specific application, a tissue scaffold of the present invention, fabricated as described above, can be used as a spinal implant 800 as depicted in FIG. 9 and FIG. 10. Referring to FIG. 9 and FIG. 10, the spinal implant 800 includes a body 810 having a wall 820 sized for engagement within a space S between adjacent vertebrae V to maintain the space S. The device 800 is formed from bioactive glass fibers that can be formed into the desired shape using extrusion methods to form a cylindrical shape that can be cut or machined into the desired size. The wall 820 has a height h that corresponds to the height H of the space S. In one embodiment, the height h of the wall 820 is slightly larger than the height H of the intervertebral space S. The wall 820 is adjacent to and between a superior engaging surface 840 and an inferior engaging surface 850 that are configured for engaging the adjacent vertebrae V as shown in FIG. 10.

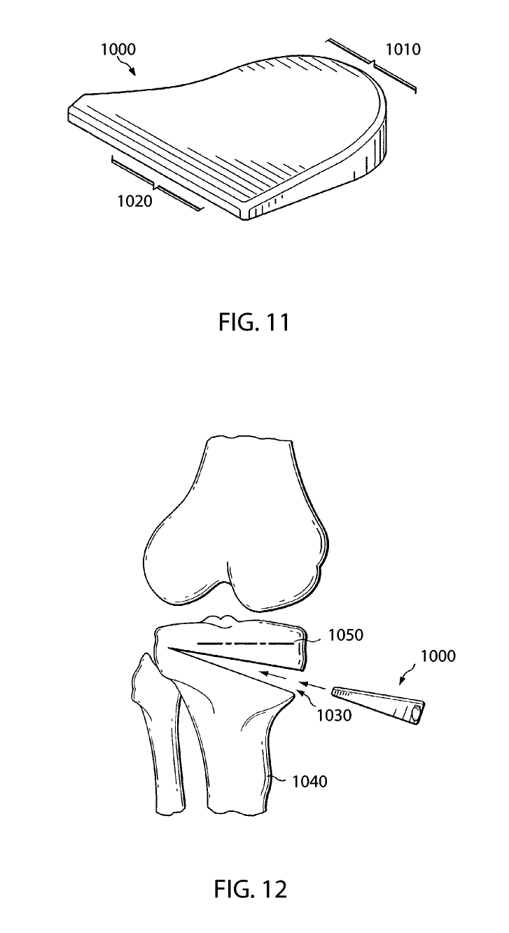

In another specific application, a tissue scaffold of the present invention, fabricated as described above, can be used as an osteotomy wedge implant 1000 as depicted in FIG. 11 and FIG. 12. Referring to FIG. 11 and FIG. 12, the osteotomy implant 1000 may be generally described as a wedge designed to conform to an anatomical cross section of, for example, a tibia, thereby providing mechanical support to a substantial portion of a tibial surface. The osteotomy implant is formed from bioactive glass fibers bonded and fused into a porous material that can be formed from an extruded rectangular block, and cut or machined into the contoured wedge shape in the desired size. The proximal aspect 1010 of the implant 1000 is characterized by a curvilinear contour. The distal aspect 1020 conforms to the shape of a tibial bone in its implanted location. The thickness of the implant 1000 may vary from about five millimeters to about twenty millimeters depending on the patient size and degree of deformity. Degree of angulation between the superior and inferior surfaces of the wedge may also be varied.

FIG. 12 illustrates one method for the use of the osteotomy wedge implant 1000 for realigning an abnormally angulated knee. A transverse incision is made into a medial aspect of a tibia while leaving a lateral portion of the tibia intact and aligning the upper portion 1050 and the lower portion 1040 of the tibia at a predetermined angle to create a space 1030. The substantially wedge-shaped implant 1000 is inserted in the space 1030 to stabilize portions of the tibia as it heals into the desired position with the implant 1000 dissolving into the body as herein described. Fixation pins may be applied as necessary to stabilize the tibia as the bone regenerates and heals the site of the implant.

Generally, the use of a resorbable bone tissue scaffold of the present invention as a bone graft involves surgical procedures that are similar to the use of autograft or allograft bone grafts. The bone graft can often be performed as a single procedure if enough material is used to fill and stabilize the implant site. In an embodiment, fixation pins can be inserted into the surrounding natural bone, and/or into and through the resorbable bone tissue scaffold. The resorbable bone tissue scaffold is inserted into the site and fixed into position. The area is then closed up and after a certain healing and maturing period, the bone will regenerate and become solidly fused.

The use of a resorbable bone tissue scaffold of the present invention as a bone defect filler involves surgical procedures that can be performed as a single procedure, or multiple procedures in stages or phases of repair. In an embodiment, a resorbable tissue scaffold of the present invention is placed at the bone defect site, and attached to the bone using fixation pins or screws. Alternatively, the resorbable tissue scaffold can be externally secured into place using braces. The area is then closed up and after a certain healing and maturing period, the bone will regenerate to repair the defect.

A method of filling a defect in a bone includes filling a space in the bone with a resorbable tissue scaffold comprising bioactive fibers bonded into a porous matrix, the porous matrix having a pore size distribution to facilitate in-growth of bone tissue; and attaching the resorbable tissue scaffold to the bone.

A method of treating an osteotomy includes filling a space in the bone with a resorbable tissue scaffold comprising bioactive fibers bonded into a porous matrix, the porous matrix having a pore size distribution to facilitate in-growth of bone tissue; and attaching the resorbable tissue scaffold to the bone.

A method of treating a structural failure of a vertebrae includes filling a space in the bone with a resorbable tissue scaffold comprising bioactive fibers bonded into a porous matrix, the porous matrix having a pore size distribution to facilitate in-growth of bone tissue; and attaching the resorbable tissue scaffold to the bone.

A method of fabricating a synthetic bone prosthesis includes mixing bioactive fiber with a binder, a pore former and a liquid to provide a plastically formable batch; kneading the formable batch to distribute the bioactive fiber with the pore former and the binder, the formable batch a homogeneous mass of intertangled and overlapping bioactive fiber; forming the formable batch into a desired shape to provide a shaped form; drying the shaped form to remove the liquid; heating the shaped form to remove the binder and the pore former; and heating the shaped form to a bond formation temperature using a primary heat source and a secondary heat source to form bonds between the intertangled and overlapping bioactive glass fiber.

In an embodiment, the present invention discloses the use of precursors to form a porous matrix having a bioactive composition through a chemical reaction that leads to the transformation of one set of chemical substances (the precursors) to another chemical substance (the bioactive composition). The reaction forms at elevated temperatures that is sustained over a period of time.

In an embodiment, the present invention discloses use of fibers bonded into a porous matrix having a bioactive composition, the porous matrix having a pore size distribution to facilitate in-growth of bone tissue for the treatment of a bone defect.

In an embodiment, the present invention discloses use of fibers bonded into a porous matrix having a bioactive composition, the porous matrix having a pore size distribution to facilitate in-growth of bone tissue for the treatment of an osteotomy.

In an embodiment, the present invention discloses use of fibers bonded into a porous matrix having a bioactive composition, the porous matrix having a pore size distribution to facilitate in-growth of bone tissue for the treatment of a structural failure of various parts of a spinal column.

The present invention has been herein described in detail with respect to certain illustrative and specific embodiments thereof, and it should not be considered limited to such, as numerous modifications are possible without departing from the spirit and scope of the appended claims.

What is claimed is:

1. A method of fabricating a synthetic bone prosthesis comprising:
mixing a glass fiber with a bonding agent, a pore former, and a liquid to provide a plastically formable batch, the glass fiber and the bonding agent having a composition that is a precursor to a bioactive composition;
mixing the plastically formable batch to distribute the glass fiber with the bonding agent and the pore former, to provide a formable batch of a homogeneous mass, the glass fiber being arranged in an overlapping and intertangled relationship;
forming the formable batch into a desired shape to provide a shaped form;
drying the shaped form to remove substantially all the liquid;
removing the pore former; and
heating the shaped form to react the glass fiber with the bonding agent to form a porous fiber scaffold having the bioactive composition.

2. The method according to claim 1 wherein the bonding agent comprises a calcium oxide.

3. The method according to claim 1 wherein the bonding agent comprises a phosphate.

4. The method according to claim 1 wherein the bonding agent comprises a mixture of a calcium oxide and a phosphate.

5. The method according to claim 1 wherein the glass fiber comprises a silica glass fiber.

6. The method according to claim 1 wherein the glass fiber comprises calcium-silicate fiber with a calcium oxide content less than 30% by weight.

7. The method according to claim 1 wherein the glass fiber comprises a phosphate glass fiber.

8. The method according to claim 1 wherein the bonding agent comprises a coating on the glass fiber.

9. A method of fabricating a synthetic bone prosthesis comprising:
mixing at least two precursors to provide a uniform mixture, at least one of the at least two precursors in a fiber form; and
heating the uniform mixture to react the at least two precursors to form a bioactive composition, the bioactive composition having a fibrous structure.

10. The method according to claim 9 wherein the precursor in fiber form is a silica glass fiber.

11. The method according to claim 9 wherein the precursor in fiber form is a phosphate glass fiber.

12. The method according to claim 9 wherein the at least two precursors comprise oxides of magnesium, sodium, potassium, calcium and phosphorus.

13. The method according to claim 9 wherein the at least two precursors comprise a fiber having at least one of a calcium silicate and a magnesia silicate.

14. The method according to claim 9 wherein the at least two precursors comprise calcium silicate fiber and magnesia silicate fiber.

15. A method of fabricating a bioactive synthetic bone prosthesis comprising:
providing a fiber having a composition having a low level of bioactivity;
providing a precursor of a composition;
creating a rigid porous scaffold using the fiber;
altering the composition of the rigid porous scaffold using the precursor to provide a fibrous scaffold having a level of bioactivity greater than the fiber.

16. The method according to claim 15 wherein the precursor of a composition is applied to the fiber.

17. The method according to claim 15 wherein the precursor of a composition is included in the fiber.

18. The method according to claim 15 wherein the precursor of a composition is provided after the step of creating a rigid porous scaffold.
